(19) United States
(12) Patent Application Publication
(10) Pub. No.: US 2004/0248290 A1
(43) Pub. Date: Dec. 9, 2004

(54) Culture systems for the sterile continuous cultivation of cells

(76) Inventors: Marco Riedel, Berlin (DE); Uwe Marx, Berlin (DE); Hikmat Bushnaq-Josting, Berlin (DE)

Correspondence Address: Jordan & Hamburg, 122 East 42nd Street, New York, NY, 10168, US

(21) Appl. No.: 10/488,818

(22) PCT Filed: May 31, 2003
(86) PCT No.: PCT/DE03/01826

(30) Foreign Application Priority Data
May 31, 2002 (DE) 103 25 148.01-41

(51) Int. Cl. C12N 5/06

(52) U.S. Cl. 435/325

(57) ABSTRACT

The invention relates to culture systems and methods for continuous sterile cultivation of cells in high densities and for reducing the inoculation density at the beginning of cell cultivation in bioreactors. By using biodegradable gels that release low-molecular-weight constituents as nutrients for the cell culture or semi-solid media that are diluted during the course of the culturing and thus free up culture space for colonization with cells in high density, it is possible to sharply reduce the starting cell density at the beginning of cultivation in bioreactors. Biodegradable gels used are polypeptide (block) copolymers, in part comprising poly-L-glutamine, and semi-solid media used are methylcellulose, alginates, and agaroses.

### DESCRIPTION

The invention relates to culture systems and to methods for continuous sterile cultivation of cells in high densities and for reducing the inoculation density at the beginning of cell cultivation in bioreactors. By using biodegradable gels that release low-molecular-weight constituents as nutrients for the cell culture or semi-solid media that are diluted during the course of the culturing and thereby free up culture space for colonization with cells in high density, it is possible to sharply reduce the starting cell density at the beginning of cultivation in bioreactors. Biodegradable gels used are polypeptide (block) copolymers, in part comprising poly-L-glutarnine, and semi-solid media used are methylcelluloses, alginates, and agaroses.

### CHARACTERIZATION OF PRIOR ART

Among the various different cell culturing apparatus that have been developed for a wide range of purposes, cultivating cells for the manufacture of pharmaceuticals plays an important role. Currently the cells are maintained in culture primarily using two fundamentally different methods:

1. Suspension culture in conventional sterile stirring vessel bioreactors.

2. Stationary cultivation in high cell densities that were made possible particularly by the presence of suitable separating membranes and were first described by Knazek et. al. in 1974 in U.S. Pat. No. 3,821,087. In addition to culture systems made of hollow fiber membranes, flat membranes have also been used (Scheirer and Katinger 1985, DE 3409501). However, in the aforesaid methods and apparatus, uniform nutrient supply—especially the supply of oxygen—is problematic. Neither the attempt to solve this problem using complex method steps with pressurization (1989, U.S. Pat. No. 4,804,628) nor introducing oxygen directly into the cell culture space using an additional membrane system (1986, DE 2431450, and 1995, DE 4230194) led to culture systems that could be expanded to the scale desired and in which the cells could be supplied uniformly. Membrane methods per se possess greater advantages compared to conventional suspension cultures. By operating them as perfusion cultures, they can achieve very high cell densities using a large membrane surface area per unit of volume (10⁷-10⁸ cells/mL). In addition, the cells are protected from damaging shear forces, require less nutrient media, and have a higher product concentration (Piret J M, Cooney C L. 1990: Immobilized mammalian cell cultivation in hollow fiber bioreactors, Biotechol. Adv. 8: 763-783). In addition, both suspension cells and adherent cells can be cultivated with hollow fibers (Lipman N S, Jackson L R. 1998: Hollow fibre bioreactors: an alternative to murine ascites for small scale (<1 gram) monoclonal antibody production, Res Immunol July-August; 149 (6): 571-6].

The medium space, gel, and support material components already play a role in various writings. For instance, DE 19725318 A1, DE 19540487 A1, and U.S. Pat. No. 5,736,399 A have to do with tissue engineering of 3D structures. This concerns enabling cell-to-cell interactions. In accordance with EP 666322 A1, microorganisms in the sense of bacteria are cultivated. As for direct contact plates, which are already known and can be obtained commercially, this concerns cultivation of bacteria/fungi and the subsequent microbial count.

Cultivation of cells in bioreactors always begins with inoculation. Cells are added to the bioreactor in a defined cell density. This starting cell density has a lower limit and depends on the bioreactor. It is suspected that this minimum starting cell density is necessary to perform a type of “self-conditioning” in order to then make cultivation possible. There is still much that is unclear about the essential mechanisms of self-conditioning and about the molecules responsible for this (Gstraunthaler G. et. al.: Impact of culture conditions, culture media volumes, and glucose content on metabolic properties of renal epithelial cell cultures; Are renal cells in tissue culture hypoxic? Cell. Physiol. Biochem. 9: 150-172, 1999). At the same time, the cell density that can be attained in these bioreactors has an upper limit. The limit is primarily characterized by reduced metabolite exchange of the individual cells.

### THE ESSENCE OF THE INVENTION

The object of the invention is to make possible new options for cultivating cells of high cell density and to reduce the minimum starting density (inoculation density).

This object is attained by the development of culture systems and methods for continuous sterile cultivation of cells in high densities in which the separation of the cells from the nutrient medium occurs by embedding the cells in a gel and/or semi-solid medium that is maintained by a support material. The invention makes it surprisingly possible for the cultivation of cells to be begun with very low inoculation densities of ≦10,000 cells per milliliter.

For gels, the inventive culture systems contain cross-linked polypeptides with a high glutamine portion, and for semi-solid media they contain viscous liquids, in particular methylcellulose, or liquids made of inventively produced microscopic gel particles.

Among the properties of the inventively used and if appropriate growth-promoting semi-solid medium is that it is biodegradable and/or is diluted during the course of cultivation and thus the culture space can be maximally used by the cells in high density. Another advantage of the inventively used gels and semi-solid matrices is that the low-molecular-weight constituents that are released by biodegradation can be used as nutrient media for the cell culture.

In the framework of this specification, the term “cells” refers to naturally and coincidentally degraded cells of any species and/or cells of any species that are degraded by manipulation, and the phrase “cells in high density” indicates concentrations of individual cells in sterile culture of greater than 10 million cells per milliliter.

The inventive culture systems contain a culture space with a plurality of fixed chambers and a medium space with a device for producing a variably adjustable gel/cell culture media mixture, whereby the culture space and the medium space are semi-permeably separated from one another.

The products obtained using the invention (cell constituents, viruses, and active substances produced in and by cells) are free of compounds that are atypical for the media.

Another surprising advantage of the invention is that high cell densities are obtained in one and the same culture space and thus there is no need to change to larger culture spaces.

One of the fundamental intents of the invention is to cultivate the cells in the reactor across several orders of magnitude in the cell concentration such that it is not necessary to change to larger reactor systems.

In cell culture systems, actual cell reproduction occurs between the minimum starting density (inoculation density) and the maximum achievable cell density. Thus, in a bioreactor, the cell densities can be increased one-hundredfold on average and at constant volume.

In the field of tissue engineering, providing sufficient cells or tissue material for successful cultivation is often problematic.

For production-scale cultivation, very high numbers of cells are necessary, and these must be cultivated from a pool (working cell bank, WCB). The cells must be expanded by a factor of 1,000,000. This is only possible when the cells are cultivated in at least three bioreactor systems that have been adjusted to one another in terms of increasing the scale.

It is enormously advantageous when it is possible to reduce drastically the starting cell density because this means multiple bioreactors systems can be omitted. Not having to transfer cells from one bioreactor system to another is equivalent to reducing culture duration, minimizing risk (danger of contamination), and savings in expenditures for equipment. In the case of stirring vessel bioreactors, this minimum starting cell density is generally 1×10⁵ cells/mL, but for hollow fiber bioreactors it is 2×10⁶ cells/mL.

The invention uses gels and/or semi-solid matrices to solve the problems described in the foregoing and to reduce the minimum starting cell density (inoculation density).

The term “gels” is understood to include cross-linked polypeptides with a high glutamine portion or cross-linked alginates; semi-solid media are viscous liquids with a viscosity that is at least 20-100 times greater than that of water or even greater, such as for instance solutions of methylcellulose (MC) or agar (note: not agaroses, see http://www.mgm.musin.de/projekte/elba/algen/algen13.htm), as well as liquids that comprise microscopic gel particles.

In the framework of this specification, gel, semi-solid medium, or growth-promoting semi-solid medium indicates a medium in which individual cells can be deposited and caused to reproduce. Such gels are preferably cross-linked polypeptides with a high glutamine portion or cross-linked alginates, whereas semi-solid media comprise viscous liquids, preferably methylcellulose, or liquids made of microscopic gel particles.

The semi-solid media also contain support materials. A support material (e.g. a hollow fiber membrane) fixes the gel or semi-solid medium in the culture space and facilitates unimpeded metabolism between environment and gel or semi-solid medium. Suitable materials are flat membranes, tubular membranes, and woven netting. The membranes comprise polymers e.g. polysulfones, polyether sulfones, or polycarbonates, and also comprise polyesters of the group of polyalkylene terephthalates, especially polyethylene terephthalate.

Tubular membranes are hollow fiber membranes that can be produced for instance by spinning processes or extrusion processes or that comprise flat membranes that have been rolled into a tube and fused.

Woven netting is fleece in any desired geometry. Tubular membranes and fleeces can also comprise the same materials as flat membranes.

Filling the cell culture space in a bioreactor with a gel or semi-solid matrix leads to a higher viscosity in the cell culture space. If this cell culture space is inoculated with cells, then they are held “fast” in the matrix. The matrix reduces the metabolism speed about the cells so that a microenvironment can develop. With an optimum matrix, each cell can build its own microenvironment so that it is possible to cultivate a single cell.

During the course of the cell expansion, the gel is gradually replaced by cells. This process is supported by the use of proteolytic degradable gels such as polypeptide (block) copolymers (A.P.Nowak, V. Breedveld, L. Pakstis, B. Ozbas, D. J. Pine, D. Pochan, and T. J. Deming, Rapidly recovering hydrogel scaffolds from self-assembling diblock copolypeptide amphiphiles, Nature 417 (2002), pp. 424-428), whereby the proteolytic degradation correlates positively with the concentration of cell proteases, that is, with the cell count. If these polypeptide (block) copolymers are built up to an overwhelming portion of poly-L-glutamine, the amino acid L-glutamine occurs during biodegradation; apart from glucose, it acts as the main source of nutrients for animal cells.

One inventive novel polypeptide (block) copolymer is obtained by reacting poly-L-glutamic acid in oxalyl chloride and introducing ammonia (Example 2.3). It comprises poly-L-glutamine and poly-L-glutamic acid, e.g. 89 mol % poly-L-glutamine and 11 mol % poly-L-glutamic acid.

Instead of the inventively used gels based on polypeptides, methylcelluloses can also be used when their employment in the cell culture has been checked. In cell culture, methylcelluloses are currently used primarily in cloning; they are commercially available for this application and are therefore easy to obtain (Kanakura, Y., Sonoda, S., Nakano, T., Fujita, J., Kuriu, A., Asai, H., and Kitamura, Y., Formation of mast cell colonies in methylcellulose by mouse skin cells and development of mucosal-like mast cells from the Pathol., 129:168-176, 1987. cloned cells in the gastric mucosa of W/Wv mice. Am. J.). Until now they have not been used for a semi-solid matrix for building a microenvironment about the individual cell and thus have not been a requirement for reducing inoculation density. This is a novel use of semi-solid matrices in accordance with the invention.

Among the inventive new semi-solid media are gels that are obtained from human serum albumin (HSA) and glutardialdehyde by cross-linking. Once the reaction has concluded, water is added to the hardened gels and the gels are comminuted using a dispersion device (Example 3). After processing, the resulting gel particles are between 10 μm and 100 μm in size.

Cross-linking to gels and their subsequent comminution leads to products that make it possible to mix them with cells and introduce them to a support material (e.g. a hollow fiber membrane). The effect is then the same as for methylcellulose. The cells remain:

a) fixed in the culture space, that is, they cannot sediment, and

b) metabolism is hampered by diffusion, and there is better self-conditioning evidenced in the lower cell count that can be used.

Plant cells or animal cells (mammalian cells) can be cultivated with the inventive method.

The inventive method and the inventive culture systems (apparatus) are extremely suitable for obtaining proteins, active substances, and pharmaceuticals.

In the inventive cell cultivation there is only diffusive transport of nutrients—there is no active transport between the cells.

The features of the invention follow from the elements of the claims and from the specification, whereby both individual features and multiple features in the form of combinations represent advantageous embodiments for which this application seeks protection. The essence of the invention comprises a combination of known elements (general constituents of bioreactors) and novel elements (semi-solid media, maintained by support materials) that affect one another and that provide in their overall effect an advantage of use and the desired success, which is that for the first time an option has been created for clearly reducing the minimum starting cell density (inoculation density) when cultivating cells in high density.

The inventive use of the culture system and/or the methods lies in the cultivation of cells in high density and in reducing the starting cell density at the beginning of cultivation in bioreactors. It furthermore lies in obtaining cell products, cell constituents, viruses, or active substances, as well as in obtaining pharmaceuticals and in manufacturing diagnostic products. It furthermore lies in the fact that gels and/or semi-solid matrices are used to build a microenvironment about the individual cell and thus as a requirement for reducing the inoculating density they are diluted during the course of the culturing and thereby free up culture space for colonization with cells in high densities. It also lies in the fact that they release low-molecular-weight constituents as nutrients for the cell culture.

This is also where the inventive use of methylcelluloses is found.

In the aforesaid documents, DE 19725318 A1, DE 19540487 A1, and U.S. Pat. No. 5,736,399 A, which concern tissue engineering of 3D structures, the issue is not (as in the instant invention) the deposit of a very small number of cells in the culture space and expansion to high cell densities; rather, the issue is facilitating cell-to-cell interactions. The difference from EP 666322 A1, in which cultivation of bacteria/fungi and subsequent microbial count is stressed, is that the gels only act as nutrient and moisture reservoirs and the bacteria do not have to be introduced into the gel or into a semi-solid medium, but rather remain on the surface.

The invention shall be explained in greater detail without being restricted to the examples.

### EXEMPLARY EMBODIMENTS

### Example 1

Cages are produced that are made of a permeable membrane and a plastic housing. The permeable membrane is a flat membrane made of polyethylene terephthalate with identical pores with a pore diameter of 0.4 μm. The plastic housing comprises polycarbonate.

Each of the cages contains one 1-mL culture space. They are held submerged in a solution that contains nutrients so that the cells can be supplied nutrients through the membrane.

The cages are filled with a 2% methylcellulose/medium mixture (MC) and inoculated with cells. The inoculation concentrations are 5×10³, 5×10⁴, 5×10⁵, and 5×10⁶ cells/mL (c/mL). These concentrations are shown in Table 1 as Day 0 cell concentrations.

TABLE 1
Mean (n = 3) of cell concentrations (hybridoma cells) in each of the
cages:

| Trial | Day | Vital [c/mL] | Dead [c/mL] |
|---|---|---|---|
| 5 × 10³ cells/mL in 2% methylcell- | Day 0 | 5 × 10³ | 1 × 10³ |
| ulose | Day 4 | 33 × 10³ | 35 × 10³ |
| 5 × 10⁴ cells/mL in 2% methylcell- | Day 0 | 5 × 10⁴ | 1 × 10⁴ |
| ulose | Day 4 | 31.5 × 10⁴ | 16.5 × 10⁴ |
| 5 × 10⁵ cells/mL in 2% methylcell- | Day 0 | 5 × 10⁵ | 1 × 10⁵ |
| ulose | Day 4 | 2.9 × 10⁵ | 3.8 × 10⁵ |
| 5 × 10⁶ cells/mL in 2% methylcell- | Day 0 | 5 × 10⁶ | 1 × 10⁶ |
| ulose | Day 4 | 3.5 × 10⁶ | 1.9 × 10⁶ |
| 5 × 10³ cells/mL | Day 0 | 5 × 10³ | 1 × 10³ |
| | Day 4 | 0 | 0 |
| 5 × 10⁴ cells/mL | Day 0 | 5 × 10⁴ | 1 × 10⁴ |
| | Day 4 | 2.2 × 10⁴ | 3.9 × 10⁴ |
| 5 × 10⁵ cells/mL | Day 0 | 5 × 10⁵ | 1 × 10⁵ |
| | Day 4 | 4.4 × 10⁵ | 4.2 × 10⁵ |
| 5 × 10⁶ cells/mL | Day 0 | 5 × 10⁶ | 1 × 10⁶ |
| | Day 4 | 7.5 × 10⁶ | 3.4 × 10⁶ |

These data clearly indicate that the methylcellulose permitted a reduction in the minimum required inoculation concentration by a factor of 10-100.

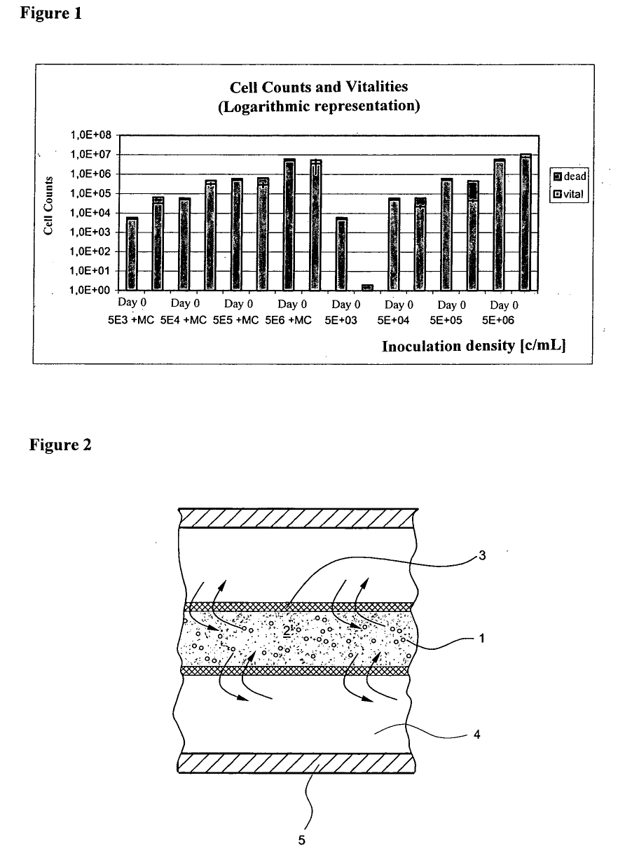

FIG. 1: Cell counts and vitality

FIG. 2:

Culture systems (5) for continuous sterile cultivation of cells in high densities and for reducing the starting cell density at the beginning of cultivation in bioreactors, in which the cells (1) are separated from the nutrient medium (4), characterized in that they contain a gel (2) or a semi-solid medium that is held by a support material (3).

### EXAMPLE 2

The polypeptide (block) copolymers are produced as follows:

2.1: Production of a monomer

0.03 mol glutamic acid and 0.011 mol triphosgene are reacted in 70 mL THF (tetrahydrofurane) at 50° C.

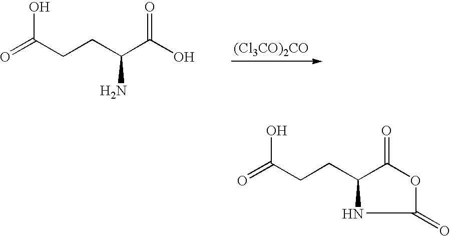

The N-carboxylic acid anhydride of glutamic acid forms. The solvent is completely drained off. Purification occurs using recrystallization from ethyl acetate. Two bands are found in the Fourier-transformed IR spectrum at 1750 and 1815 cm⁻¹ that are typical for the cyclic anhydride formed.

2.2: Production of a Homopolymer

0.01 mol of the monomer is reacted with 0.3 mmol triethylamine in 20 mL tetrahydrofurane (THF) at room temperature (RT). The reaction lasts 7 days. By adding ethyl acetate, the polymer is completely precipitated and washed. The poly-L-glutamic acid formed is water-soluble.

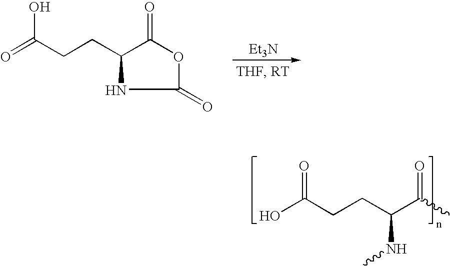

2.3: Functionalizing the Polymer

1.0 g poly-L-glutamic acid is added to 50 mL oxalyl chloride. Oxalyl chloride is also the solvent. The reaction to the acid chloride causes the polymer to go into solution. After 48 hours at room temperature the solvent is drained off and the remaining polymer is dissolved in 50 mL THF. Gaseous ammonia is introduced into the polymer solution, whereby the polymer begins to precipitate. After two hours of adding the gas, the precipitate is removed, washed with THF, dried, and dialyzed in a 10-kD dialysis tube in water. Elementary analysis indicated that 89 mol % of the polymer comprises poly-L-glutamine and 11 mol % comprises poly-L-glutamic acid. The determination of the molecular weight using membrane osmometry yielded a mean count of 53000 g/mol. In water there was a dynamic viscosity of 1020 mPa's (milli Pascal seconds) at 20° C.

### Example 3

### Semi-Solid Media

6·10⁻⁵ mol of the human serum albumin protein (HSA) are reacted with 8.4·10⁻⁴ mol glutardialdehyde in a beaker in 10.37 mL water at room temperature. After 48 hours the hardened gel is transferred to a soxhlet device and extracted with water for 12 hours at 100° C.

Water equivalent to five times the volume of the extracted gel is added thereto and comminuted into gel particles using a dispersion device. After 10 minutes of processing, gel particles result that are between 10 μm and 100 μm in size. The solidity of the gel particles, as well as their size, can be adjusted using the selection of the HSA concentration, the glutardialdehyde concentration, the dispersion tool, and the duration of processing.

The gel particles were autoclaved. In one cell culture trial, 900 μL of the comminuted gel were blended with 100 μL of a cell suspension that had a concentration of 1·10⁴ cells/mL. The resulting cell density was thus 1·10³ cells/mL.

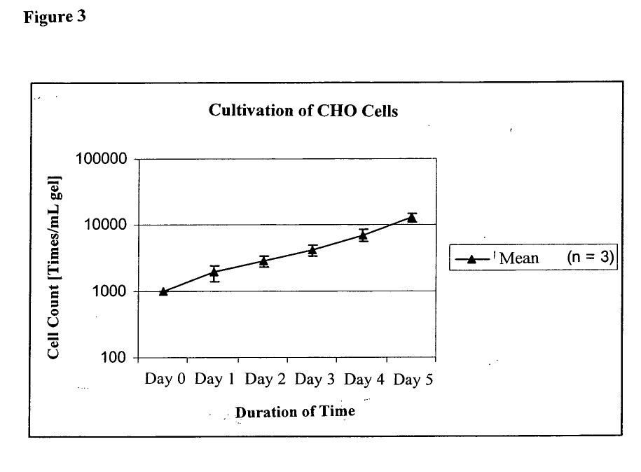

500 μL of this suspension are added to a 24-well cell culture plate. This corresponds to a gel bed height of approx. 3 mm. The cell-containing gel in the cell culture plate is coated with 50 μL fresh medium and incubated at 37° C. in the incubator (5 vol. % CO₂). The cell count is performed daily. The cell count in the gel increases with culture duration (see FIG. 3).

### Example 4

Gel particles of HSA were autoclaved. In one cell culture trial, 900 μL of the comminuted gel were blended with 100 μL of a cell suspension that had a concentration of 5·10⁴ cells/mL. The resulting cell density was thus 5·10³ cells/mL.

500 μL of this suspension are added to a 24-well cell culture plate. This corresponds to a gel bed height of approx. 3 mm. The cell-containing gel in the cell culture plate is coated with 500 μL fresh medium and incubated at 37° C. in the incubator (5 vol. % CO₂). The old medium is exchanged for fresh medium every three or four days. The supernatant of the old medium is used to determine the concentration of the antibody that was produced by the cells during the course of cultivation.

The cell count is performed on the day the medium is changed (see Table 2).

TABLE 2
Mean (n = 3) of cell concentration during cultivation of CHO cells in
microscopic gel particles of HSA and antibody concentration
in the cell culture supernatant

| | | | Expansion factor | |
|---|---|---|---|---|
| | | | for vital cells | Antibody |
| | | | (cell count on | concentra- |
| | Vital | Dead | Day x the cell | tion |
| Day | [cells/mL] | [cells/mL] | count on Day 0) | [μg/mL] |
| Day 0 | 4.8 · 10³ | 0.2 · 10³ | 1 | — |
| Day 3 | 1.5 · 10⁴ | 4.8 · 10³ | 3 | 0.21 |
| Day 7 | 9.4 · 10⁴ | 1.6 · 10⁴ | 20 | 1.30 |
| Day 10 | 3.3 · 10⁵ | 2.9 · 10⁴ | 69 | 4.13 |
| Day 14 | 1.3 · 10⁶ | 2.1 · 10⁵ | 271 | 20.3 |
| Day 17 | 3.3 · 10⁶ | 6.8 · 10⁵ | 692 | 45.5 |
| Day 21 | 8.8 · 10⁶ | 2.2 · 10⁶ | 1833 | 158.7 |

Legend (FIG. 2)

1 Cells

2 Gel/semi-solid medium

3 Support material

4 Supply medium

5 Culture system

Legend for Figures

FIG. 1: Cell counts and vitalities/ratio of cell counts to inoculation density

FIG. 2: Culture systems for continuous sterile cultivation of cells in high densities and for reducing the starting cell density at the beginning of cultivation in bioreactors

FIG. 3: Increase in cell count with culture duration during cultivation of CHO cells

(CHO—Chinese hamster ovary)

1. Culture system for continuous sterile cultivation of cells in high densities and for reducing starting cell density at inception of cell cultivation in bioreactors, in which said cells are separated from a nutrient medium, said system comprising one selected from the group consisting of:
a gel comprising cross-linked polypeptides having a high glutamine portions; and
a semi-solid medium comprising at least one selected from the group consisting of viscous liquids and microscopic gel particle-containing liquids.

2. Culture system according to claim 1, further comprising at least one selected from the group consisting of:
a polypeptide (block) copolymer;
poly-L-glutamine;
an alginate; agar; and
a methylcellulose.

3. Culture system according to claim 1, further comprising a support material for said selected gel or semi-solid medium.

4. Culture system according to claim 1, wherein said selected gel or semi-solid medium comprises one selected from the group consisting of:
a polypeptide (block) copolymer containing both poly-L-glutamine and poly-L-glutamic acid; and
an HSA glutardialdehyde gel.

5. Culture system according to claim 3, wherein said support material comprises one selected from the group consisting of: a flat membrane, a tubular membrane, and woven netting.

6. Method for continuous sterile cultivation of cells in high densities and for reducing starting cell density at inception of cell cultivation in bioreactors, in which said cells are separated from a nutrient medium, said method comprising embedding said cells in one selected from the group consisting of: a gel and a semi-solid medium, supported by a support material.

7. Method according to claim 6, wherein said selected gel or semi-solid medium comprises one selected from the group consisting of:
a polypeptide (block) copolymer containing both poly-L-glutamine and poly-L-glutamic acid;
poly-L-glutamine;
a methylcellulose; an alginate; agar and
an HSA glutardialdehyde gel.

8. Method according to claim 7, wherein said polypeptide (block) copolymer containing both poly-L-glutamine and poly-L-glutamic acid is obtained by reacting poly-L-glutamic acid in oxalyl chloride and adding ammonia.

9. Method according to claim 7, wherein said HSA glutardialdehyde gel is obtained by reacting human serum albumin (HSA) with glutardialdehyde.

10. Method according to claim 9, wherein water is added to said HSA glutardialdehyde gel, which is then comminuted to microscopic gel particles using a dispersion device.

11. Method acording to claim 6, wherein said cultivation of cells is begun with very low inoculation densities less than or equal to 10,000 cells per milliliter and said cultivation of said cells is multiplied in a single culture space.

12. (Cancelled)

13. Method according to claim 6, wherein said selected one of said gel and said semi-solid medium produces a micro-environment about an individual cell thereby reducing inoculation density.

14. (Cancelled)

15. (Cancelled)

16. Method according to claim 6, wherein said selected one of said gel and said semi-solid medium is diluted during culturing thereby freeing culture space for colonization with cells in high density.

17. Method according to claim 6, wherein said selected one of said gel and said semi-solid medium releases low-molecular-weight constituents as nutrient media for said cell culture.
